(19) United States
(12) Patent Application Publication
(10) Pub. No.: US 2025/0095152 A1
(43) Pub. Date: Mar. 20, 2025

(54) SPATIAL BINDING OF IMAGING DATA FROM MULTIPLE MODALITIES

(71) Applicant: Alcon, Inc., Fribourg (CH)

(72) Inventors: Vignesh Suresh, Woodland Hills, CA (US); Lu Yin, Keller, TX (US); Ramesh Sarangapani, Coppell, TX (US)

(73) Assignee: Alcon, Inc., Fribourg (CH)

(21) Appl. No.: 18/828,271

(22) Filed: Sep. 9, 2024

Related U.S. Application Data
(60) Provisional application No. 63/582,917, filed on Sep. 15, 2023.

(51) Int. Cl. G06T 7/00; G06T 7/33; G06V 10/75; G06V 10/82; G06V 20/70; G16H 30/40

(52) U.S. Cl. CPC G06T 7/0014; G06T 7/337; G06V 10/751; G06V 10/82; G06V 20/70; G16H 30/40; G06T 2207/10101; G06T 2207/20084; G06T 2207/20212; G06T 2207/30041; G06T 2207/30096; G06V 2201/03

(57) ABSTRACT

A system for spatially binding images from multiple modalities includes a controller having at least one processor and at least one non-transitory, tangible memory. The controller is adapted to receive first and second imaging datasets of a target site from a first and a second modality. The controller is adapted to extract a first feature set in the first imaging dataset, via a first neural network. A second feature set is extracted from the second imaging dataset, via a second neural network. Feature pairs are generated by matching respective datapoints in the first feature set and the second feature set. The controller is adapted to determine a coordinate transformation between the feature pairs and generate at least one spatially bound image of the target site based in part on the coordinate transformation.

### CROSS REFERENCE TO RELATED APPLICATION

The present application claims the benefit of priority to U.S. Provisional Application No. 63/582,917 filed Sep. 15, 2023, which is hereby incorporated by reference in its entirety.

### INTRODUCTION

The disclosure relates generally to blending imaging data from multiple modalities. More specifically, the disclosure relates to spatially binding imaging data from multiple modalities. Various imaging modalities are commonly employed throughout the world to image various parts of the human body. Each of these imaging modalities brings a different set of information to the table. In order to maximize the available information, a synthesis of the respective information provided by various imaging modalities is desirable. However, it is not a trivial matter to blend the captures that are made in multiple domains for a user.

### SUMMARY

Disclosed herein is a system for spatially binding imaging data from multiple modalities. The system includes a controller having at least one processor and at least one non-transitory, tangible memory on which instructions are recorded. The controller is adapted to receive a first imaging dataset of a target site from a first modality and a second imaging dataset of the target site from a second modality. The controller is adapted to extract a first feature set extracted from the first imaging dataset, via a first neural network. A second feature set is extracted from the second imaging dataset, via a second neural network. Feature pairs are generated by matching a respective datapoint in the first feature set with the respective datapoint in the second feature set. The controller is adapted to determine a coordinate transformation between the feature pairs and generate at least one spatially bound image of the target site based in part on the coordinate transformation.

The target site may be an eye. In some embodiments, the first feature set is a limited set that is not representative of information captured by the first modality, and the second feature is a representation of an ocular region captured by the second modality. The controller may be configured to select at least one region of interest in the first imaging dataset, the first feature set being extracted from the at least one region of interest. The first imaging dataset may include data obtained by scanning a plurality of source wavelengths.

In one embodiment, the first modality is multispectral imaging, and the second modality is optical coherence tomography (“OCT”). Here, the first imaging dataset is captured by an OCT device, the target site being an eye. The spatially bound image extends a peripheral portion of the first imaging dataset, enabling visualization of one or more structures posterior to an iris of the eye. In another embodiment, the first modality is fluorescent angiography, and the second modality is optical coherence tomography. The first neural network may be a multilayer perceptron. The second neural network may be a convolutional neural network.

In some embodiments, the first feature set includes the respective feature points indicative of degenerative disease. In some embodiments, the first imaging dataset includes a plurality of scans. The controller may be configured to identify and isolate a pathological region as being within one of the plurality of scans or in-between two of the plurality of scans. The controller is configured to add at least one annotation over the at least one spatially bound image, the at least one annotation indicating the pathological region. In some embodiments, the controller is adapted to selectively transfer respective annotations made on the first imaging dataset to the second imaging dataset, and from the second imaging dataset to the first imaging dataset.

The above features and advantages and other features and advantages of the present disclosure are readily apparent from the following detailed description of the best modes for carrying out the disclosure when taken in connection with the accompanying drawings.

### BRIEF DESCRIPTION OF DRAWINGS

FIG. 1 is a schematic illustration of a system for spatially binding imaging data from multiple modalities, the system having a controller;

FIG. 2 is a schematic flowchart for a method executable by the controller of FIG. 1;

FIG. 3 is a schematic diagram of an example architecture employable by the system of FIG. 1;

FIG. 4 is a schematic illustration of an example imaging dataset obtained from multispectral imaging;

FIG. 5 is a schematic illustration of an example image obtained by optical coherence tomography; and

FIG. 6 is a schematic view of an example spatially bound image generated by the system of FIG. 1.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### DETAILED DESCRIPTION

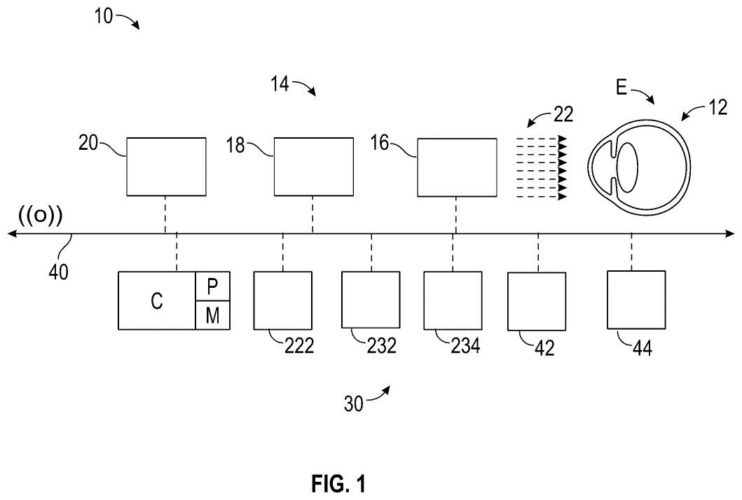

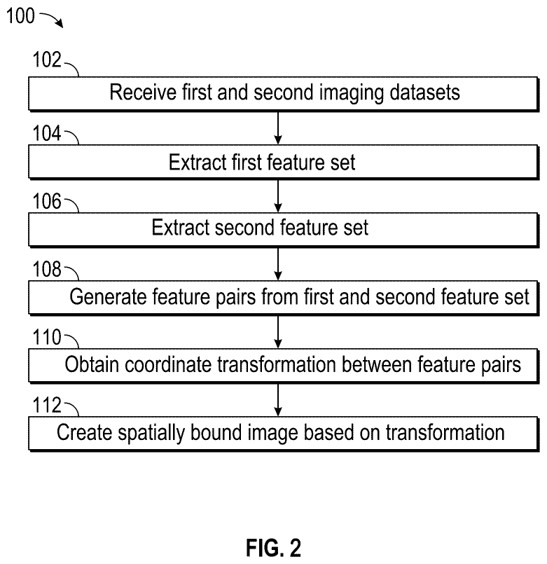

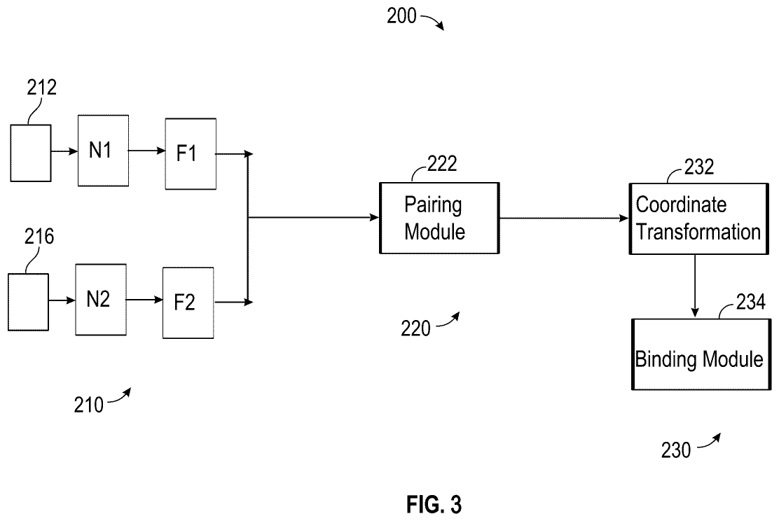

Referring to the drawings, wherein like reference numbers refer to like components, FIG. 1 schematically illustrates a system 10 having a controller C with at least one processor P and at least one memory M (or non-transitory, tangible computer readable storage medium) on which instructions are recorded for executing method 100 for spatially binding imaging data of a target site 12. Method 100 is shown in and described below with reference to FIG. 2. The imaging data is obtained from multiple modalities 14. An example architecture 200 employable by the system 10 is shown in FIG. 3 and described below.

The target site 12 in the embodiment shown is an eye E. An ocular region captured by different imaging modalities provides different spatial information and other characteristics, depending on the resolution, depth, type of source used, source wavelength and other characteristics of the imaging modality. The system 10 enhances the visualization of the target site 12 by coherently binding the imaging data that is obtained from multiple modalities 14, including a first modality 16 and a second modality 18. In some embodiments, a third modality 20 may be employed to obtain a third imaging dataset. The coherent spatial binding includes both topographical and depth information. The system 10 may learn and improve over time, and the data may be shared across sites.

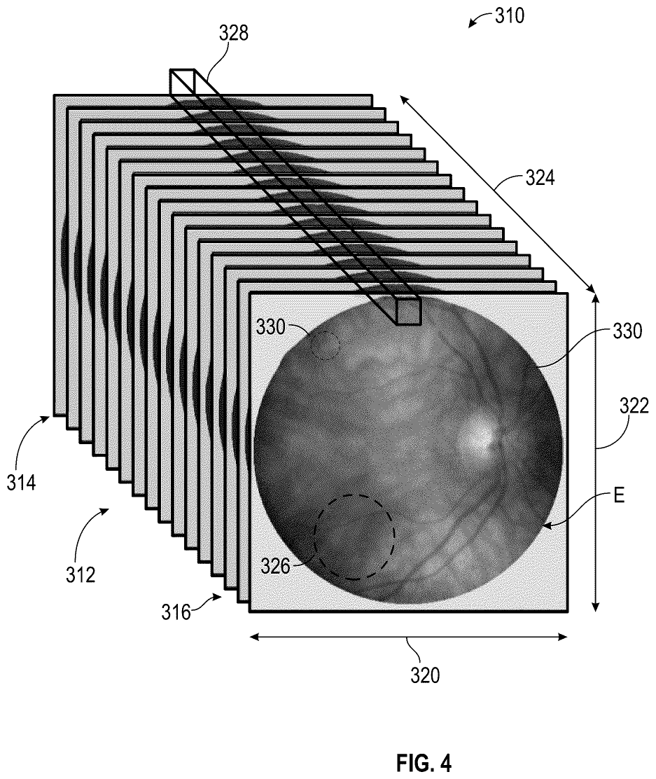

The multiple modalities 14 may include multispectral imaging. Multispectral imaging employs an apparatus with multiple monochromatic light sources, such as LEDs, to illuminate the eye 12 at wavelengths ranging from about 550 nm to 950 nm. The reflected light is collected by an image sensor (not shown). The different frames of the spectral reflectance are obtained by scanning the source wavelengths. An example imaging dataset 310 from multispectral imaging is shown in FIG. 4 and described below.

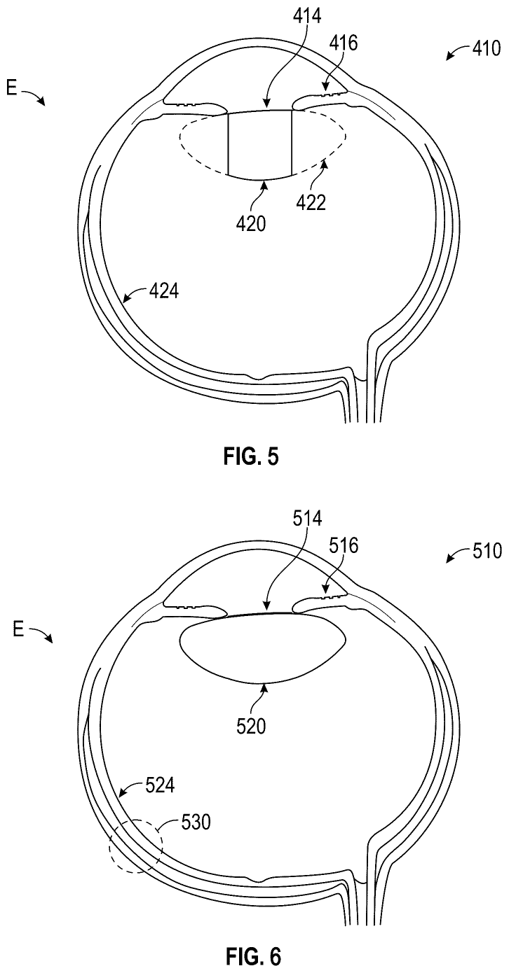

The multiple modalities 14 may include optical coherence tomography (“OCT” hereinafter), employing an OCT device. The OCT device has an array of laser beams 22 which may cover the span or width of the eye E. In one example, the OCT device is an high definition swept-source OCT imaging device. The OCT device may use time domain, frequency domain, or other suitable spectral encoding, and may use single point, parallel, or other type of scanning pattern. The OCT device may take many different forms and include multiple and/or alternate components. An example original image 410 from optical coherence tomography is shown in FIG. 5 and described below.

The multiple modalities 14 may include fluorescent angiography, which includes the use of a fluorescent dye and a specialized camera. For example, sodium fluorescein may be introduced into the systemic circulation of a patient, and the eye 12 may be illuminated with light at a specific wavelength, e.g., 490 nanometers. An angiogram is obtained by capturing the fluorescent green light that is emitted by the dye. It is understood that the multiple modalities 14 may include ultrasound bio microscopy, fundus imaging, and other modalities available to those skilled in the art.

Referring to FIG. 1, the controller C may selectively execute a plurality of modules 30, such as a feature pairing module 222, a coordinate transformation module 232 and a binding module 234. The plurality of modules 30 may be embedded in, or be otherwise accessible to, the controller C. The plurality of modules 30 may be a part of a remote server or cloud unit accessible to the controller C via a network 40.

Referring now to FIG. 3, an example architecture 200 employable by the system 10 is shown. The system 10 may be executed in three phases as shown in FIG. 3, a first phase 210 of feature selection, a second phase 220 of feature pairing, and a third phase 230 of spatial binding. As described below, the controller C is adapted to receive a first imaging dataset of the target site 12 from a first modality 16 and a second imaging dataset of the target site 12 from a second modality 18. In the first phase 210, the first imaging data set 212 is transmitted to a first neural network N1. Similarly, the second imaging data set 216 is transmitted to a second neural network N2. In the first phase 210, the first neural network N1 and the second neural network N2 respectively extract a first feature set F1 and a second feature set F2, via the first neural network N1 and second neural network N2, respectively.

In the second phase 220 shown in FIG. 3, a feature pairing module 222 may be executed to pair or match each respective feature point in the first feature set F1 with a corresponding feature point in the second feature set F2. The outcome of the feature pairing module 222 is a pair of features, e.g., for every unique feature identified on the first imaging dataset, there will be a corresponding feature in the second imaging dataset.

In the third phase 230 shown in FIG. 3, a coordinate transformation module 232 may be executed to determine a coordinate transformation between the feature pairs. The controller C may be adapted to generate at least one spatially bound image of the target site 12, via execution of a binding module 234. The spatially bound image combines the first imaging dataset and the second imaging dataset through the coordinate transformation. The spatially bound image of the target site 12 may be shown on a display 44, as shown in FIG. 1.

Blending the datasets obtained from the different systems in a coherent manner aids diagnosis in both the pre-operative and intra-operative stage. The system 10 enables the transfer of annotations made on one image to another image after binding them spatially, assisting a surgeon in marking various features interchangeably.

The various components of the system 10 may be configured to communicate via the network 40, shown in FIG. 1. The network 40 may be a bi-directional bus implemented in various ways, such as for example, a serial communication bus in the form of a local area network. The local area network may include, but is not limited to, a Controller Area Network (CAN), a Controller Area Network with Flexible Data Rate (CAN-FD), Ethernet, WIFI, Bluetooth™ and other forms of data connection. Other types of connections may be employed.

The controller C may be configured to receive and transmit data through an input device 42 that is user operable. The input device 42 may be installed on a laptop, tablet, desktop or other electronic device. The input device 42 may include a touch screen interface, a keyboard, joystick, mouse, foot switch and other devices. The input device 42 may be a mobile application on a smartphone. The circuitry and components of a mobile application (“apps”) available to those skilled in the art may be employed. The input device 42 may include an integrated processor and integrated memory.

Referring now to FIG. 2, an example flowchart of the method 100 is shown. Method 100 may be embodied as computer-readable code or instructions stored on and partially executable by the controller C of FIG. 1. Method 100 need not be applied in the specific order recited herein and may be dynamically executed. Furthermore, it is to be understood that some steps may be eliminated. As used herein, the terms ‘dynamic’ and ‘dynamically’ describe steps or processes that are executed in real-time and are characterized by monitoring or otherwise determining states of parameters and regularly or periodically updating the states of the parameters during execution of a routine or between iterations of execution of the routine.

Beginning at block 102, the method 100 includes receiving a first imaging dataset of the target site 12 from a first modality 16 and a second imaging dataset of the target site 12 from a second modality 18. In one embodiment, the first modality 16 is multispectral imaging, and the second modality 18 is optical coherence tomography (“OCT” hereinafter). In another embodiment, the first modality 16 is fluorescent angiography, and the second modality 18 is OCT.

Referring now to FIG. 4, an example imaging dataset 310 from multispectral imaging is shown. The imaging dataset 310 includes a plurality of scans 312 of an eye E. In other words, the imaging dataset 310 includes multiple two-dimensional images stacked together, extending over a horizontal axis 320 and a vertical axis 322. The imaging dataset 310 obtained from multispectral imaging covers a range 324 of wavelength and may cover 20-40 spectral bands. Thus, scan 314 may cover a shorter wavelength (e.g., 550 nm) and scan 316 may cover a longer wavelength (e.g., 850 nm). Each data set contains a set of images because the peaks in each spectrum may be spatially mapped. Therefore, each scan or two-dimensional image has both spatial and spectral information, i.e., each spatial locus has an associated spectrum when viewed across the available wavelengths.

Referring to FIG. 5, an example original image 410 obtained from optical coherence tomography is shown. The original image 410 shows the pupil 414, the iris 416, retina 424, and the lens 420 of an eye E. OCT imaging does not capture the peripheral portion 422 of the lens 420 that is behind the iris 416. This is because the illuminating lasers used in OCT imaging cannot penetrate across the iris 416. However, OCT imaging techniques provide high resolution and a non-contact scanning method that is convenient in terms of patients' compliance and comfort in daily clinical settings. For example, the OCT imaging is performed in the sitting position, and takes a relatively short amount of time.

Proceeding to block 104, the method 100 includes extracting a first feature set F1 from the first imaging dataset, via a first neural network N1. In some embodiments, the controller C is adapted to select at least one region of interest, such as first region of interest 326 in the first imaging dataset 310. The first feature set F1 is then extracted from within the region of interest 326, via the first neural network N1. In one example, the region of interest 326 includes feature points indicative of degenerative disease, such as a tumor. The region of interest may be captured in the form of a cuboid 328, covering each of the plurality of scans 312.

In some embodiments, the first neural network N1 is a multilayer perceptron, which is a feedforward artificial neural network that generates a set of outputs from a set of inputs. As understood by those skilled in the art, a multilayer perceptron is characterized by several layers of input nodes connected as a directed graph between the input and output layers.

Advancing to block 106, the controller C is adapted to extract a second feature set from the second imaging dataset, via a second neural network N2. In some embodiments, the second neural network N2 incorporates a convolutional neural network (CNN)-based or other deep learning techniques available to those skilled in the art. Convolutional neural networks are a specialized type of artificial neural networks that use convolution in place of general matrix multiplication in at least one of their layers. The second neural network N2 may be specifically designed to process pixel data for image recognition and processing. It is understood that the first neural network N1 and the second neural network N2 may include any other type of neural network available to those skilled in the art.

In some embodiments, a user may select or pre-program various parameters for feature extraction, including a desired number of feature points per frame, a maximum number of feature points per frame and a minimum value of the spacing between the feature points. Additionally, the controller C may be programmed to adaptively calculate a respective optimal quality level for the features in the region of interest. The quality level is a parameter characterizing the minimally accepted quality of an image feature point. In some embodiments, the quality level is a normalized number between 0 and 1. The feature point with a respective score or quality measuring less than the quality level may be rejected.

Proceeding to block 108, the method 100 includes generating feature pairs by matching a respective datapoint in the first feature set F1 with the respective datapoint in the second feature set F2. For example, the first feature set F1 may include 80 feature points and the second feature set may include 100 features. The controller C may be able to successfully identify a match for a portion of the first feature set F1, such as for example, 60 features of the 80 features in the first feature set F1. The feature matching may be made based upon common features, such as edges, points, corners, and landmarks. The feature matching may be made based upon regions with relatively high contrast and other factors.

Advancing to block 110, the controller C is adapted to determine a coordinate transformation between each of the feature pairs, transforming coordinates from the frame of reference of the first imaging dataset to the frame of reference of the second imaging dataset. The transformation may be based on a deterministic system, using one or more predefined equations or relations. transformation may be a learning-based method using a neural network. Because the transformation involves redundant mapping, the data may be filtered for more precise results.

Proceeding to block 112, the method 100 includes generating at least one spatially bound image of the target site 12 based in part on the coordinate transformation. FIG. 5 illustrates an example OCT image of the eye E. FIG. 6 is a schematic view of an example spatially bound image 510. The spatially bound image 510 shows the pupil 514, the iris 516, retina 524, and the lens 520 of the eye E. The system 10 enables reconstruction of the complete image of the lens 520 of FIG. 6, including the peripheral portion 422 absent in the original image 410 of FIG. 5.

The controller C may be configured to identify and isolate a pathological region 330 as being within one of the plurality of scans 312 (of the first imaging modality or the second imaging modality) or in-between two of the plurality of scans 312. The controller C may be configured to display or annotate or draw in an overlay dashed line or sphere (see FIG. 6) at the location of the pathological region 330. The pathological region 530 may be depicted with onscreen pixels of different brightness or color compared to the surroundings. By combining the imaging datasets as described above, and further highlighting or annotating the area where the pathological region 530 is found, diagnosis and treatment is aided.

In some embodiments, the controller C is adapted to selectively transfer annotations made on the first imaging dataset to the second imaging dataset, and annotations made on the second imaging dataset to the first imaging dataset. As noted above, the imaging dataset from multispectral imaging may cover 20-40 spectral bands. The longer wavelengths (e.g., 670 nm) enable the visualization of the deeper layers of the retina, the retinal pigment epithelium, and the choroid. The information from different layers of the eye with a plurality of source wavelengths enables the detection of melanin, which is a pigment in the retinal pigment epithelial. Areas where melanin is detected in the imaging dataset (obtained from multispectral imaging) may be marked within the same and transferred over into the spatially bound image 510. Additionally, the longer wavelengths allow the retinal vasculature to be mapped by detecting oxygenation of blood. Areas where oxygenation is detected in the imaging dataset (obtained from multispectral imaging) may be marked within the same and transferred over into the spatially bound image 510.

In summary, the system 10 generates at least one spatially bound image of a target site 12 based in part on a coordinate transformation between feature pairs from multiple modalities. The feature pairs are generated by matching a respective datapoint in the first feature set F1 with the respective datapoint in the second feature set F2. The system 10 has several technical advantages. First, the outcome of the spatial binding module 244 provides a blending of multiple images which will enhance visualization for ophthalmologists. Additionally, the outcome of the feature pairing module 222 may be used to select one feature in the first imaging modality and automatically predict the corresponding one in the second imaging modality.

The controller C of FIG. 1 includes a computer-readable medium (also referred to as a processor-readable medium), including a non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic medium, a CD-ROM, DVD, other optical medium, a physical medium, a RAM, a PROM, an EPROM, a FLASH-EEPROM, other memory chip or cartridge, or other medium from which a computer can read.

Look-up tables, databases, data repositories or other data stores described herein may include various kinds of mechanisms for storing, accessing, and retrieving various kinds of data, including a hierarchical database, a set of files in a file storage system, an application database in a proprietary format, a relational database energy management system (RDBMS), etc. Each such data store may be included within a computing device employing a computer operating system such as one of those mentioned above and may be accessed via a network in one or more of a variety of manners. A file system may be accessible from a computer operating system and may include files stored in various formats. An RDBMS may employ the Structured Query Language (SQL) in addition to a language for creating, storing, editing, and executing stored procedures, such as the PL/SQL language mentioned above.

The flowchart shown in the FIGS. illustrates an architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by specific purpose hardware-based systems that perform the specified functions or acts, or combinations of specific purpose hardware and computer instructions. These computer program instructions may also be stored in a computer-readable medium that can direct a controller or other programmable data processing apparatus to function in a particular manner, such that the instructions stored in the computer-readable medium produce an article of manufacture including instructions to implement the function/act specified in the flowchart and/or block diagram blocks.

The numerical values of orders (e.g., of quantities or conditions) in this specification, including the appended claims, are to be understood as being modified in each respective instance by the term “about” whether or not “about” actually appears before the numerical value. “About” indicates that the stated numerical value allows some slight imprecision (with some approach to exactness in the value; about or reasonably close to the value; nearly). If the imprecision provided by “about” is not otherwise understood in the art with this ordinary meaning, then “about” as used herein indicates at least variations that may arise from ordinary methods of measuring and using such orders. In addition, disclosure of ranges includes disclosure of each value and further divided ranges within the entire range. Each value within a range and the endpoints of a range are hereby disclosed as separate embodiments.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

1. A system comprising:
a controller having at least one processor and at least one non-transitory, tangible memory on which instructions are recorded for a method of spatially binding imaging data, execution of the instructions by the processor causing the controller to:
receive a first imaging dataset of a target site from a first modality and a second imaging dataset of the target site from a second modality;
extract a first feature set from the first imaging dataset, via a first neural network;
extract a second feature set from the second imaging dataset, via a second neural network;
generate feature pairs by matching a respective datapoint in the first feature set with the respective datapoint in the second feature set;
determine a coordinate transformation between the feature pairs; and
generate at least one spatially bound image of the target site based in part on the first imaging dataset, the second imaging dataset and the coordinate transformation.

2. The system of claim 1, wherein the target site is an eye.

3. The system of claim 1, wherein:
the first feature set is a limited set that is not representative of information captured by the first modality; and
the second feature is a representation of an ocular region captured by the second modality.

4. The system of claim 1, wherein the controller is configured to select at least one region of interest in the first imaging dataset, the first feature set being extracted from the at least one region of interest.

5. The system of claim 1, wherein the first imaging dataset includes data obtained by scanning a plurality of source wavelengths.

6. The system of claim 1, wherein the first modality is multispectral imaging, and the second modality is optical coherence tomography (“OCT”).

7. The system of claim 6, wherein:
the first imaging dataset is captured by an OCT device, the target site being an eye; and
the at least one spatially bound image extends a peripheral portion of the first imaging dataset, enabling visualization of one or more structures posterior to an iris.

8. The system of claim 6, wherein the first neural network is a multilayer perceptron.

9. The system of claim 8, wherein the second neural network is a convolutional neural network.

10. The system of claim 1, wherein the first modality is fluorescent angiography, and the second modality is optical coherence tomography.

11. The system of claim 1, wherein the first feature set includes the respective feature points indicative of degenerative disease.

12. The system of claim 1, wherein:
the first imaging dataset includes a plurality of scans;
the controller is configured to identify and isolate a pathological region as being within one of the plurality of scans or in-between two of the plurality of scans; and
the controller is configured to add at least one annotation over the at least one spatially bound image, the at least one annotation indicating the pathological region.

13. The system of claim 1, wherein the controller is adapted to selectively transfer respective annotations made on the first imaging dataset to the second imaging dataset, and from the second imaging dataset to the first imaging dataset.

14. A system comprising:
a controller having at least one processor and at least one non-transitory, tangible memory on which instructions are recorded for a method of spatially binding imaging data, execution of the instructions by the processor causing the controller to:
receive a first imaging dataset of an eye from a first modality and a second imaging dataset of the eye from a second modality, the first modality being multispectral imaging, and the second modality being optical coherence tomography;
select at least one region of interest in the first imaging dataset;
extract a first feature set from the at least one region of interest, via a first neural network;
extract a second feature set from the second imaging dataset, via a second neural network;
generate feature pairs by matching a respective datapoint in the first feature set with the respective datapoint in the second feature set; and
determine a coordinate transformation between the feature pairs and generate at least one spatially bound image of the eye based in part on the coordinate transformation.

15. The system of claim 14, wherein:
the first feature set is a limited set that is not representative of information captured by the first modality; and
the second feature is a representation of an ocular region captured by the second modality.

16. The system of claim 14, wherein the first neural network is a multilayer perceptron, and the second neural network is a convolutional neural network.

17. The system of claim 14, wherein:
the first imaging dataset includes a plurality of scans;
the controller is configured to identify and isolate a pathological region as being within one of the plurality of scans or in-between two of the plurality of scans; and
the controller is configured to add at least one annotation over the at least one spatially bound image, the at least one annotation indicating the pathological region.

18. The system of claim 14, wherein the controller is adapted to selectively transfer respective annotations made on the first imaging dataset to the second imaging dataset, and from the second imaging dataset to the first imaging dataset.
